(19) United States
(12) Patent Application Publication
(10) Pub. No.: US 2021/0220055 A1
(43) Pub. Date: Jul. 22, 2021

(54) ANATOMIC OR PHYSIOLOGICAL STATE DATA CLUSTERING

(71) Applicant: Brainlab AG, München (DE)

(72) Inventors: Chris Maier, München (DE); Oleksii Diubankov, Munich (DE); Martin Krenner, Unterhaching (DE); Nikoleta Mouriki, Munich (DE); Mario Degenhardt, Munich (DE)

(21) Appl. No.: 16/098,020

(22) PCT Filed: Sep. 22, 2017
(86) PCT No.: PCT/EP2017/074066

§ 371 Date: Oct. 31, 2018

(51) Int. Cl. A61B 34/20; A61B 34/30; A61N 1/36; A61N 2/00; A61B 5/00; A61N 5/10; G16H 10/60; G16H 50/70; G16H 20/40

(52) U.S. Cl. CPC A61B 34/20; A61B 34/30; A61N 1/3606; A61N 2/006; A61B 5/7475; A61B 2034/107; A61N 5/10; G16H 10/60; G16H 50/70; G16H 20/40; A61B 5/4842

(57) ABSTRACT

Disclosed is a computer-implemented method for processing patient data so that it is clustered. The patient data may comprise imagesets and other treatment-related data (e.g. planned objects, trajectories) in a way that each cluster of data describes one specific anatomic or physiological state of the patient. The clustered patient data is filtered to automatically determine its relevance for a user.

The present invention relates to a computer-implemented method for processing patient data, a corresponding computer program, a non-transitory program storage medium storing such a program and a computer for executing the program, as well as a medical system comprising an electronic data storage device and the aforementioned computer.

### TECHNICAL BACKGROUND

The present invention is directed to clustering available patient data (such as imagesets and other treatment related data, e.g. planned objects, trajectories) in a way that each cluster of data describes one specific anatomic or physiological state of the patient and within that the most relevant data for the user is detected automatically. In known approaches, available data is ordered for each patient according to its creation date or the device creating the data (e.g. magnetic resonance scanner). Imagesets can be grouped according to its creation time since they belong to the same study. All data is displayed to the user. Clustering data is a way of grouping data in a way that it is visibly associated and separated from other data.

The present invention has the object of allowing efficient selection of patient data for treatment planning, controlling a medical device and/or treatment.

The present invention can be used for providing structured data enabling therapeutic or surgical procedures e.g. in connection with a system for image-guided radiotherapy.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE PRESENT INVENTION

In the following, a short description of the specific features of the present invention is given which shall not be understood to limit the invention only to the features or a combination of the features described in this section.

The disclosed method encompasses processing of patient data which may comprise at least one of image data and non-image data. The processing includes clustering the patient data according to at least one anatomic or physiological state of the patient which defines the patient's state at a specific point in time. In one example, the patient data can also comprise pathological data. The clustered data (e.g. each cluster) is then filtered for example to determine whether at least part of the patient data contained in a cluster shall be removed (specifically, removed from the cluster and/or the data to be further processed). This may for example be used to reduce the quantity of data and/or extract the relevant data to be output to a user (for example, in the framework of planning a medical procedure). If the filtering procedure does not comprise any filter applicable to the patient data (e.g. to the type of patient data), the data filtering is stopped.

### GENERAL DESCRIPTION OF THE PRESENT INVENTION

In this section, a description of the general features of the present invention is given for example by referring to possible embodiments of the invention.

In general, the invention reaches the aforementioned object by providing, in a first aspect, a computer-implemented medical method of processing patient data. The method comprises executing, on at least one processor of at least one computer (for example at least one computer being part of the navigation system), the following exemplary steps which are executed by the at least one processor.

In a (for example first) exemplary step, patient data is acquired, for example from a DICOM server or a picture archiving and communication system (PACS), which describes medical information about a patient. In examples of the method according to the first aspect, the medical information comprises or consists of at least one of the following medical data sets, as defined here or above:
a screenshot or a videoclip which has been electronically generated and/or stored;
medical image information (e.g. a medical image such as at least one of a computed x-ray tomography, a magnetic resonance tomography, a sonography, or a radiography which describes for example an anatomical body part of the patient) represented in a DICOM format;
medical non-image information, representing for example a region of the body part, for example a voxel object defining a (e.g. strict) subset of medical image information;
treatment plan information usable for example in radiotherapy or surgery;
trajectory information describing a trajectory for placing an instrument relative to a patient's body;
patient documentation describing a medical state of the patient;
a point, for example annotation point or a landmark;
a fusion (i.e. an image fusion) or a registration (i.e. an image registration);
further non-DICOM data.

In a (for example second) exemplary step, cluster data is determined based on the patient data, wherein the cluster data describes at least one cluster defining a clustering of the medical information for at least one anatomic or physiological state of the patient. In an example, each cluster describes an association of at least one, for example strict, subset of the medical information with at least one, for example exactly one anatomic or physiological state. It is possible that two clusters describe the same anatomic or physiological state. If e.g. the below-described time gap rule applies, there are two clusters, because it is assumed that they describe two different anatomic or physiological states. For example, the anatomic or physiological state encompasses a characterization of the patient's state at a specific point in time in consideration of a pathology of the patient, for example in regard of at least one of the patient's anatomy (e.g. whether a tumour or brain shift is present or not) or the patient's physiology (e.g. a physiological indicator such as at least one of the patient's ECG, EEG, at least one blood value, blood pressure, body temperature, level of at least one certain hormone, or at least one immunological function). The pathology for example encompasses a tumour disease and the anatomic or physiological state is defined for example by at least one of a size of a tumour or the degree of metastasis spread.

In a (for example third) exemplary step, filtered data is determined based on the cluster data, wherein the filtered data describes a result of applying a filter rule to the medical information for at least one cluster described by the cluster data, wherein the result describes at least one of the following:
an indication as to whether the filter rule can be applied to the medical information;
an indication as to whether the medical information shall be removed from the cluster;
an indication as to whether the medical information shall be used in a medical environment.

Using the medical information in the medical environment includes in examples at least one of the following:
outputting (e.g. displaying) the medical information on a display device;
deep brain stimulation or transcranial magnetic brain stimulation (for example, for determining a time development of the anatomical or physiological state due to deep brain stimulation or transcranial magnetic brain stimulation); or
controlling a medical device on the basis of the result of applying the filter rule, for example controlling the operation of at least one of:
   a beam source or patient support unit of a radiation treatment apparatus;
   an imaging unit (such as at least one of a three-dimensional scanner or a thermal camera) of a radiation treatment apparatus;
   a robot for conducting a medical procedure;
   a display device of an image-guided navigation system to display the medical information (e.g. medical image information)—the image-guided navigation system may operate on the principle of tracking the position of at least one reflective or electromagnetically resonating marker device;
   a deep brain stimulation electrode or a transcranial magnetic stimulation device.

In an example, of the method according to the first aspect, cluster rule data is acquired which describes at least one clustering rule for clustering the patient data, and the cluster data is determined based on the cluster rule data. Clustering means for example a grouping of a part of the patient data in a way that it is visibly associated and separated from another part of the patient data. The at least one clustering rule may in examples include at least one rule for clustering the medical information based on (e.g. according to) at least one of the following criteria:
a point in time at which the medical information was generated;
a time interval between points in time at which (e.g. at least one of strict or disjunct) subsets of the medical information were generated;
manual input by a user for defining a cluster extent;
a type of the medical information;
an anatomic region to which the medical information relates;
the anatomic and physiological state (which may be determined e.g. by automatic image analysis of medical image information contained in the medical information e.g. using an atlas), for example a pathology;
data generated by other programs (which may use or not use the medical data).

In a further exemplary step of the method according to the first aspect, atlas data is acquired which describes an image-based generic model (i.e. an atlas) of an anatomical body part of the patient. The atlas can be patient-specific or not patient-specific. The atlas data is for example compared to the patient data (for example, medical image information contained in the patient data which describes an image of the anatomical body part) to determine for example the anatomical or physiological state by determining a deviation between the image of the anatomical body part described by the medical image information and the image-based description of the anatomical body part described by the atlas data. Such a deviation may for example be used to determine the presence of a brain shift or a pathology such as a tumour. Alternatively or additionally, the atlas data may be compared to the medical image information to determine a position of the anatomical body part in the medical image defined by the medical image information. The position so determined may be used as a basis for determining the cluster data, for example for clustering the patient data according to an anatomical or physiological state which is defined by at least that position.

The patient data (specifically, the medical image information) and the atlas data may be compared by applying an image fusion algorithm such as a rigid or elastic image fusion algorithm to the medical image information and the atlas data. The atlas can in one example be an electro-physiologic atlas which describes at least one of a sensitivity or functionality of at least one region of the anatomical body part. The at least one region may in a more specific example be a functional region of the brain so that the comparison between the atlas data and the medical image information may be used to determine a position at which an electrode for deep brain stimulation or of a stimulation device for transcranial magnetic stimulation shall be placed to stimulate the functional region. The position so determined may be used as a basis for determining the cluster data, for example for clustering the patient data according to an anatomical or physiological state which is defined by at least that position.

In an example of the method according to the first aspect, filter rule data is acquired which describes at least one filter rule for filtering at least one of the cluster data or the patient data, and the filtered data is determined based on the filter rule data. In examples, the at least one filter rule includes at least one rule for filtering the medical information based on (e.g. according to) at least one of the following criteria:
a point in time at which the medical information was generated;
an anatomical or physiological state of the patient, for example a pathology;
a clinical workflow of which the patient shall be the subject;
an anatomic region to which the medical information relates;
a medical imaging modality to which the medical information relates or with which it was generated;
a size (such as at least one of image count or pixel dimensions) of the digital (e.g. electronic) dataset comprising the medical information;
manual user input (for example, a dataset can be rated as important or not relevant by a user which leads to showing or hiding the dataset in the cluster;
automatic verification of manual input.

In an example of the method according to the first aspect, selection data is determined based on the filtered data, wherein the selection data describes a selection of the medical information clustered in the at least one cluster described by the cluster data for example for the use in the medical environment. For example, selection rule data is acquired which describes at least one selection rule for selecting the selection of the medical information clustered in the at least one cluster described by the cluster data. The selection data is then determined for example based on the selection rule data. In an example, the selection rule includes at least one rule for selecting the medical information to which the filter rule has been applied. The selection rule may be suitable for selecting the medical information based on (e.g. according to) at least one of the following criteria:
a type of the medical information (e.g. a treatment plan);
the point in time with which a cluster is associated (e.g. an indication which cluster is associated with the most recent and/or latest point in time);
an envisaged medical procedure to be carried out on the patient (such as radiotherapy or surgery).

The selection data is then in one example optionally transferred to a (for example external) data managing application to be stored for further use. The further use in examples encompasses using the medical information as described above, specifically using it on the basis of the selection data. The medical information may in other examples be used on the basis of the selection data without transferring the selection data to the data managing application.

In an example of the method according to the first aspect, at least one of the at least one clustering rule, the at least one filter rule and the at least one selection rule is generated by machine learning which is executed e.g. on the medical information.

In a second aspect, the invention is directed to a computer program which, when running on at least one processor (for example, a processor) of at least one computer (for example, a computer) or when loaded into at least one memory (for example, a memory) of at least one computer (for example, a computer), causes the at least one computer to perform the above-described method according to the first aspect. The invention may alternatively or additionally relate to a (physical, for example electrical, for example technically generated) signal wave, for example a digital signal wave, carrying information which represents the program, for example the aforementioned program, which for example comprises code means which are adapted to perform any or all of the steps of the method according to the first aspect.

In a third aspect, the invention is directed to a non-transitory computer-readable program storage medium on which the program according to the fourth aspect is stored.

In a fourth aspect, the invention is directed to at least one computer (for example, a computer), comprising at least one processor (for example, a processor) and at least one memory (for example, a memory), wherein the program according to the fourth aspect is running on the processor or is loaded into the memory, or wherein the at least one computer comprises the computer-readable program storage medium according to the fifth aspect.

In a fifth aspect, the invention is directed to a medical system, comprising:

a) the at least one computer according to the fourth aspect;
b) at least one electronic data storage device storing at least the patient data; and
c) a medical device for carrying out a medical procedure on the patient,
wherein the at least one computer is operably coupled to
the at least one electronic data storage device for acquiring, from the at least one data storage device, at least the patient data, and
the medical device for issuing a control signal to the medical device for controlling the operation of the medical device on the basis of the result of applying the filter rule to the medical information and, as far as the program causes the at least one computer to determine the selection data, the selected medical image information.

In an example of the system according to the fifth aspect, the medical device comprises a radiation treatment apparatus comprising a treatment beam source and a patient support unit (such as at least one of a patient bed or a headrest). The at least one computer is then operably coupled to the radiation treatment apparatus for issuing a control signal to the radiation treatment apparatus for controlling, on the basis of the result of applying the filter rule to the medical information and, as far as the program causes the at least one computer to determine the selection data, the selected medical information, at least one of the operation of the treatment beam source (e.g. for controlling emission of a treatment beam of ionising radiation) or the position of the patient support unit.

In an example of the system according to the fifth aspect, the medical device comprises a robot for conducting a medical procedure. The at least one computer is then operably coupled to the robot for issuing a control signal to the robot for controlling, on the basis of the result of applying the filter rule to the medical information and, as far as the program causes the at least one computer to determine the selection data, the selected medical information, the operation of the robot.

### Definitions

In this section, definitions for specific terminology used in this disclosure are offered which also form part of the present disclosure.

The method in accordance with the invention is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

The expression “acquiring data” for example encompasses (within the framework of a The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing the data by means of a computer and for example within the framework of the method in accordance with the invention. The meaning of “acquiring data” also for example encompasses the scenario in which the data are received or retrieved by the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

In the field of medicine, imaging methods (also called imaging modalities and/or medical imaging modalities) are used to generate image data (for example, two-dimensional or three-dimensional image data) of anatomical structures (such as soft tissues, bones, organs, etc.) of the human body. The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT), as mentioned by Wikipedia. The image data thus generated is also termed “medical imaging data”. Analytical devices for example are used to generate the image data in apparatus-based imaging methods. The imaging methods are for example used for medical diagnostics, to analyse the anatomical body in order to generate images which are described by the image data. The imaging methods are also for example used to detect pathological changes in the human body. However, some of the changes in the anatomical structure, such as the pathological changes in the structures (tissue), may not be detectable and for example may not be visible in the images generated by the imaging methods. A tumour represents an example of a change in an anatomical structure. If the tumour grows, it may then be said to represent an expanded anatomical structure. This expanded anatomical structure may not be detectable; for example, only a part of the expanded anatomical structure may be detectable. Primary/high-grade brain tumours are for example usually visible on MRI scans when contrast agents are used to infiltrate the tumour. MRI scans represent an example of an imaging method. In the case of MRI scans of such brain tumours, the signal enhancement in the MRI images (due to the contrast agents infiltrating the tumour) is considered to represent the solid tumour mass. Thus, the tumour is detectable and for example discernible in the image generated by the imaging method. In addition to these tumours, referred to as “enhancing” tumours, it is thought that approximately 10% of brain tumours are not discernible on a scan and are for example not visible to a user looking at the images generated by the imaging method.

Image fusion can be elastic image fusion or rigid image fusion. In the case of rigid image fusion, the relative position between the pixels of a 2D image and/or voxels of a 3D image is fixed, while in the case of elastic image fusion, the relative positions are allowed to change.

In this application, the term “image morphing” is also used as an alternative to the term “elastic image fusion”, but with the same meaning.

Elastic fusion transformations (for example, elastic image fusion transformations) are for example designed to enable a seamless transition from one dataset (for example a first dataset such as for example a first image) to another dataset (for example a second dataset such as for example a second image). The transformation is for example designed such that one of the first and second datasets (images) is deformed, for example in such a way that corresponding structures (for example, corresponding image elements) are arranged at the same position as in the other of the first and second images. The deformed (transformed) image which is transformed from one of the first and second images is for example as similar as possible to the other of the first and second images. Preferably, (numerical) optimisation algorithms are applied in order to find the transformation which results in an optimum degree of similarity. The degree of similarity is preferably measured by way of a measure of similarity (also referred to in the following as a “similarity measure”). The parameters of the optimisation algorithm are for example vectors of a deformation field. These vectors are determined by the optimisation algorithm in such a way as to result in an optimum degree of similarity. Thus, the optimum degree of similarity represents a condition, for example a constraint, for the optimisation algorithm. The bases of the vectors lie for example at voxel positions of one of the first and second images which is to be transformed, and the tips of the vectors lie at the corresponding voxel positions in the transformed image. A plurality of these vectors is preferably provided, for instance more than twenty or a hundred or a thousand or ten thousand, etc. Preferably, there are (other) constraints on the transformation (deformation), for example in order to avoid pathological deformations (for instance, all the voxels being shifted to the same position by the transformation). These constraints include for example the constraint that the transformation is regular, which for example means that a Jacobian determinant calculated from a matrix of the deformation field (for example, the vector field) is larger than zero, and also the constraint that the transformed (deformed) image is not self-intersecting and for example that the transformed (deformed) image does not comprise faults and/or ruptures. The constraints include for example the constraint that if a regular grid is transformed simultaneously with the image and in a corresponding manner, the grid is not allowed to interfold at any of its locations. The optimising problem is for example solved iteratively, for example by means of an optimisation algorithm which is for example a first-order optimisation algorithm, such as a gradient descent algorithm. Other examples of optimisation algorithms include optimisation algorithms which do not use derivations, such as the downhill simplex algorithm, or algorithms which use higher-order derivatives such as Newton-like algorithms. The optimisation algorithm preferably performs a local optimisation. If there is a plurality of local optima, global algorithms such as simulated annealing or generic algorithms can be used. In the case of linear optimisation problems, the simplex method can for instance be used.

In the steps of the optimisation algorithms, the voxels are for example shifted by a magnitude in a direction such that the degree of similarity is increased. This magnitude is preferably less than a predefined limit, for instance less than one tenth or one hundredth or one thousandth of the diameter of the image, and for example about equal to or less than the distance between neighbouring voxels. Large deformations can be implemented, for example due to a high number of (iteration) steps.

The determined elastic fusion transformation can for example be used to determine a degree of similarity (or similarity measure, see above) between the first and second datasets (first and second images). To this end, the deviation between the elastic fusion transformation and an identity transformation is determined. The degree of deviation can for instance be calculated by determining the difference between the determinant of the elastic fusion transformation and the identity transformation. The higher the deviation, the lower the similarity, hence the degree of deviation can be used to determine a measure of similarity.

A measure of similarity can for example be determined on the basis of a determined correlation between the first and second datasets.

The present invention relates to the field of controlling a treatment beam. The treatment beam treats body parts which are to be treated and which are referred to in the following as “treatment body parts”. These body parts are for example parts of a patient's body, i.e. anatomical body parts.

Ionising radiation is an example of radiation emittable by the radiation treatment apparatus and is used for example for the purpose of treatment. For example, the treatment beam comprises or consists of ionising radiation. The ionising radiation comprises or consists of particles (for example, sub-atomic particles or ions) or electromagnetic waves which are energetic enough to detach electrons from atoms or molecules and so ionise them. Examples of such ionising radiation include x-rays, high-energy particles (high-energy particle beams) and/or ionising radiation emitted from a radioactive element. The treatment radiation, for example the treatment beam, is for example used in radiation therapy or radiotherapy, such as in the field of oncology. For treating cancer in particular, parts of the body comprising a pathological structure or tissue such as a tumour are treated using ionising radiation. The tumour is then an example of an anatomical body part.

In one example, atlas data is acquired which describes (for example defines, more particularly represents and/or is) a general three-dimensional shape of the anatomical body part. The atlas data therefore represents an atlas of the anatomical body part. An atlas typically consists of a plurality of generic models of objects, wherein the generic models of the objects together form a complex structure. For example, the atlas constitutes a statistical model of a patient's body (for example, a part of the body) which has been generated from anatomic information gathered from a plurality of human bodies, for example from medical image data containing images of such human bodies. In principle, the atlas data therefore represents the result of a statistical analysis of such medical image data for a plurality of human bodies. This result can be output as an image—the atlas data therefore contains or is comparable to medical image data. Such a comparison can be carried out for example by applying an image fusion algorithm which conducts an image fusion between the atlas data and the medical image data. The result of the comparison can be a measure of similarity between the atlas data and the medical image data. The atlas data comprises image information (for example, positional image information) which can be matched (for example by applying an elastic or rigid image fusion algorithm) for example to image information (for example, positional image information) contained in medical image data so as to for example compare the atlas data to the medical image data in order to determine the position of anatomical structures in the medical image data which correspond to anatomical structures defined by the atlas data.

The human bodies, the anatomy of which serves as an input for generating the atlas data, advantageously share a common feature such as at least one of gender, age, ethnicity, body measurements (e.g. size and/or mass) and pathologic state. The anatomic information describes for example the anatomy of the human bodies and is extracted for example from medical image information about the human bodies. The atlas of a femur, for example, can comprise the head, the neck, the body, the greater trochanter, the lesser trochanter and the lower extremity as objects which together make up the complete structure. The atlas of a brain, for example, can comprise the telencephalon, the cerebellum, the diencephalon, the pons, the mesencephalon and the medulla as the objects which together make up the complex structure. One application of such an atlas is in the segmentation of medical images, in which the atlas is matched to medical image data, and the image data are compared with the matched atlas in order to assign a point (a pixel or voxel) of the image data to an object of the matched atlas, thereby segmenting the image data into objects.

### DESCRIPTION OF THE FIGURES

In the following, the invention is described with reference to the appended figures which give background explanations and represent specific embodiments of the invention. The scope of the invention is however not limited to the specific features disclosed in the context of the figures, wherein

FIG. 1 illustrates a basic flow of the method according to the first aspect;

FIG. 2 illustrates a clustering rule defining a time gap between two imagesets;

FIG. 3 illustrates a manual clustering of imagesets;

FIG. 4 illustrates an exemplary implementation of the step of determining the filtered data;

FIG. 5 illustrates an addition of a voxel object to a cluster; and

FIG. 6 is a schematic illustration of the system according to the fifth aspect.

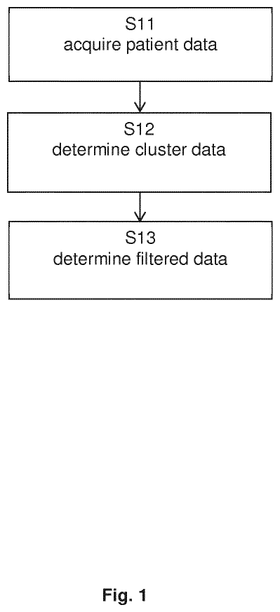

FIG. 1 illustrates the basic steps of the method according to the first aspect, in which step S11 encompasses acquiring the patient data, step S12 encompasses determining the cluster data and subsequent step S13 encompasses determining the filtered data.

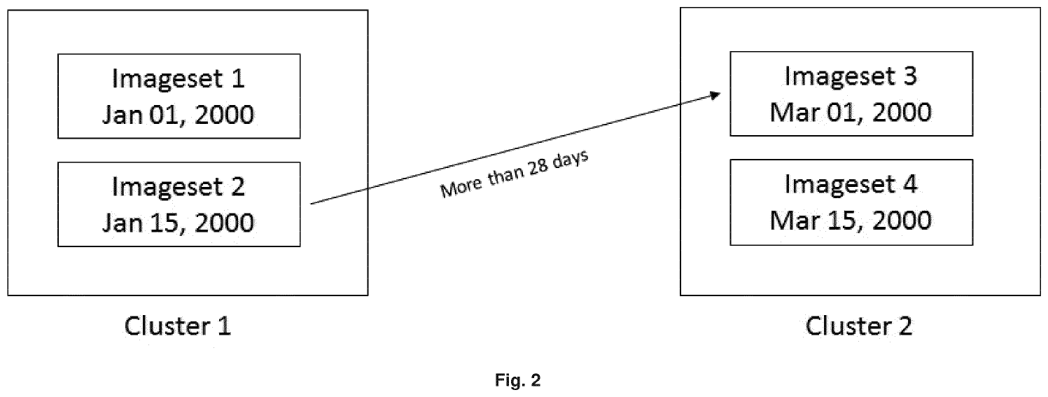

FIG. 2 describes a clustering of two imagesets (sets of image data) 1 and 2 into a first cluster 1 and two (different) imagesets 3 and 4 into a second cluster 2. A time interval being present between the points in time at which imagesets were generated is defined as a clustering rule. If such a time interval is determined for imagesets 1 to 4, a cluster boundary is defined between two imagesets for which the time interval is present, i.e. the imagesets are clustered using that boundary. The four imagesets are analysed as to whether and, if so, where a time interval of at least 28 days is present between the points in time at which the imagesets were generated. It is determined that such an interval is present between the points in time at which imageset 2 and imageset 3 were generated. The four imagesets are thus grouped into two clusters 1 and 2, wherein a time interval of at least 28 days and therefore a cluster boundary is set between the youngest imageset (imageset 2) in one cluster (cluster 1) and the oldest imageset (imageset 3) in the other cluster (cluster 2).

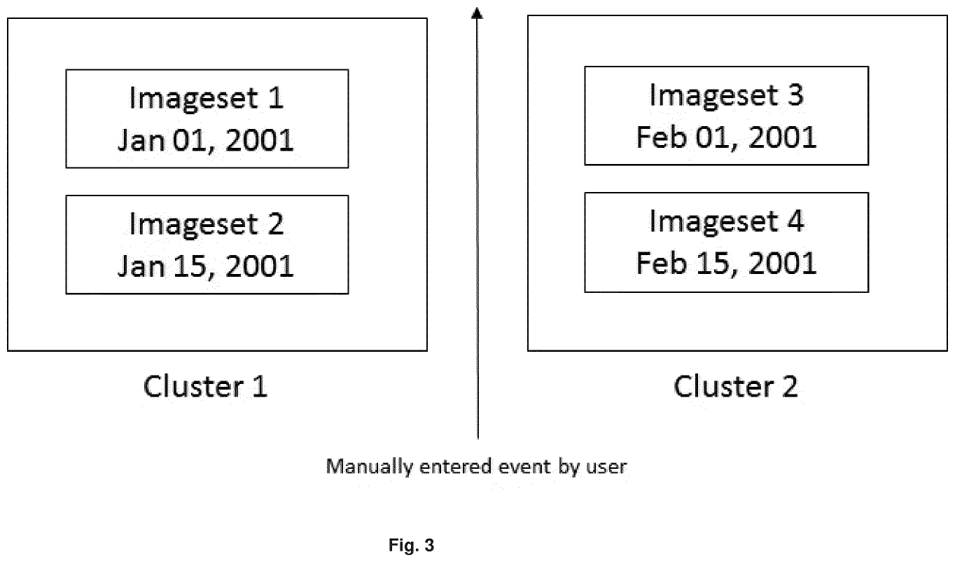

Alternatively and as illustrated in FIG. 3, the cluster boundary can be defined manually, e.g. by user input (for example, using an input device such as a keyboard, a mouse or any other pointing tool).

This procedure of clustering resembles determination of the cluster data.

The principle of clustering is that a set of clustering rules is defined where each of the rules is an algorithm that can determine for a set of data whether the data belongs to the same anatomic or physiological state of the patient. The rules takes as input all available patient data, manual input by the user and other data sources like data provided by other applications (e.g. Brainlab applications) or from an external system like a Hospital Information System (HIS). In general, an arbitrary amount of rules can be applied.

Examples of clustering rules are as follows:

Imagesets embodying the patient data are processed by rules and put into clusters as follows:
If a time distance (time interval) between two subsequent imagesets is more than a specified range (e.g. a threshold time duration such as 28 days, which may be taken as a fixed value or as a variable value; this value can be determined automatically based on available information like disease or anatomic region), the imagesets are separated into different clusters. The time period can be set according to the pathology assuming that it is long enough to introduce changes in the anatomic or physiological state of the patient.
User input splits a cluster or leads to the creation of a new one for all newer data that arrives (i.e. is generated).
Another application (working on the same set or a sub-set of the patient data, e.g. DICOM data) can generate medical information representing a change in the patient state, e.g. navigation software creates information which indicates that a change of the anatomic or physiological state of the patient has occurred.
New image data is created during a surgery and sent to the Brainlab system. The fact that new image data is created intraoperatively is an indicator that the anatomic or physiological state changes.
If subsequent imagesets are created from a different body region of the patient, the other anatomic region can be regarded as different state.
Information from external systems (e.g. Hospital information system) can be used to retrieve information (via e.g. messages according to HL7 standard) that a change of the anatomic or physiological state of the patient has occurred.
Another application (working on the same set or a sub-set of the patient data, e.g. DICOM data) can analyse the image content of the imagesets and determine that the anatomic or physiological state of the patient changed.

Non-image data embodying the patient data (such as voxel objects, trajectories, treatment plans and other data) is in examples processed by rules as follows:
Data is associated with an imageset where applicable, e.g. data describing at least one region of an anatomical body part such as voxel objects are associated with the imageset where they are segmented. The creation date of voxel object is not considered. It is put into the cluster of the imageset with which it is associated.
Treatment plans reference a set of other data. The imageset with the latest creation date referenced in the treatment plan is considered as the associated imageset and the treatment plan is put into the cluster where that imageset is put into. Alternatively, an imageset having a predetermined (but not necessarily the latest) creation date referenced in the treatment plan is considered as the associated imageset.
When a treatment plan is retrieved before all of its referenced data was retrieved, it is put on-hold to wait until all referenced data is clustered to be able to decide about the cluster it will be put into.

On the resulting set of data grouped into one cluster, filters (i.e. at least one filter) are applied. This corresponds to determining the filtered data. A filter determines for each single data item in the cluster whether it shall be displayed to the user or not or if it cannot be decided by that filter. Several filters can be applied in a row. A filter takes as input the data from the cluster and can work relative to all that available data.

The filtering of the patient data after clustering is in examples performed as follows:
All data resulting in one cluster will be filtered according to a set of filter rules.
Filtering the set of data in one cluster reduces the visible amount of data (e.g. amount of data to be displayed) to that what is most likely relevant for the user to continue treatment planning or controlling a medical device.
The result of one filter rule is the decision to leave the data in the cluster, or to remove it, or it cannot be decided yet and another filter rule must take the decision.
Filter rule may take as a decision input at least one of the following:
   Manually selected data by the user (positive or negative rating) leads to showing or hiding the data.
   A heuristic based on an information table that matches the information stored in the existing data in the cluster to that information table and selects the data that matches best (information table can e.g. contain image size, image count, image modality, anatomic region, disease information and relation between all these).
   Clinical intent (workflow): Depending on the workflow the user is currently working, imageset type and different voxel object types are relevant.

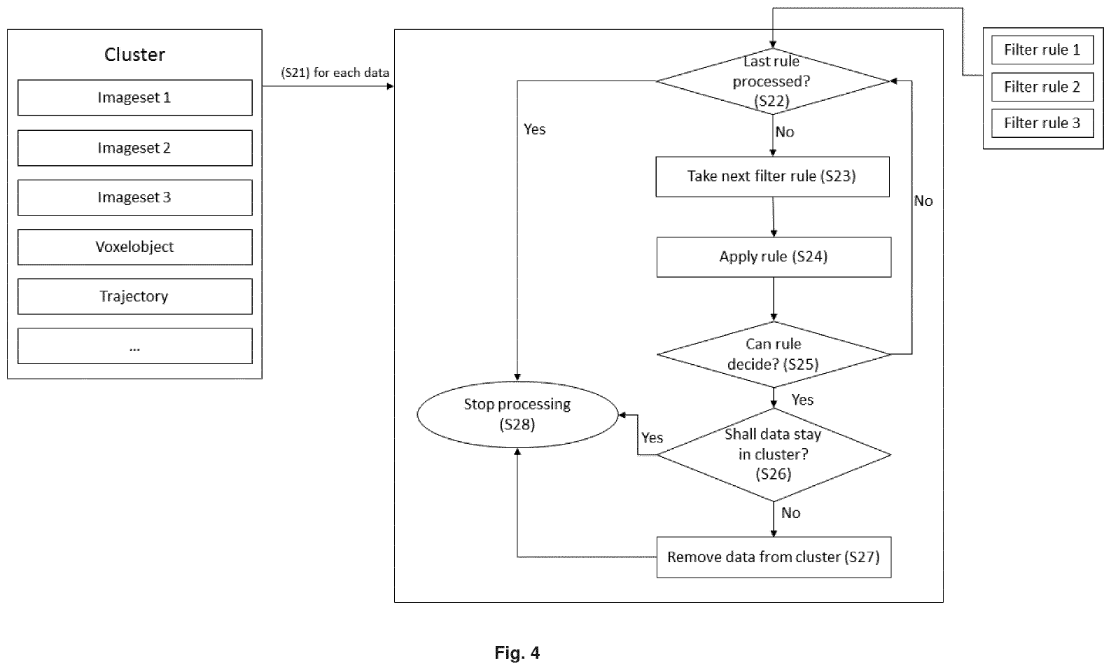

FIG. 4 illustrates an example of executing the filtering on a cluster: A set of filter rules (e.g. filter rule 1, filter rule 2, and filter rule 3) is acquired, and a cluster comprising imageset 1, imageset 2, imageset 3, a voxel object, and a trajectory, and potentially comprising medical data sets is acquired. The set of filter rules is analysed in steps S21 and S22 for each data (i.e. for each cluster) as to whether one of the filter rules has already been processed on the respective cluster. If it is determined that this is the case, the filtering procedure is stopped in step S28. If it determined that the last filter rule has not been processed with regard to the respective cluster data, the next filter rule is taken in step S23 and applied in step S24 to the cluster data. It is then determined whether the filter rule can decide, i.e. is applicable to the cluster data. If it is determined that the filter rule does not apply to the respective cluster data, the method returns to execution starting again from step S22 with selecting a next filter rule. If it is determined that the filter rule applies to the respective cluster data, it is determined in step S26 whether the data being the subject of the filter shall stay in the cluster. If it is determined in step S26, by application of the filter rule to the data, that the data shall not stay in the cluster, it is removed from the cluster in step S27. If it is determined that the data shall stay in the cluster, the filtering procedure is stopped in step S28.

Selection rules are in one example applied which decide, for a set of data, whether the data shall be selected into a temporary working set. This corresponds to determining the selection data.

According to automatic or manual selection, a subset of the clustered and filtered data is transferred to the next application. The next application can be a central data managing software storing links or an application directly controlling a medical device.

The method may also include an optional step of pre-selecting according to at least one of the following criteria:
The output of clustering rules and filtering rules determine groups of data visibly reduced to a subset of data.
On that data a selection rule can be applied which selects a specific set of data to a temporary working set used to continue working with that data in other applications. Examples of selection rules are:
   If the latest cluster (by date) contains an autosaved treatment plan, that treatment plan is selected.
   If the latest cluster contains a treatment plan that one is selected (the latest if there is more than one).
   If the latest cluster contains a treatment plan of a specific type that matches the current treatment type, this one is selected.
   If there is no treatment plan in the visible set of data in the cluster, all visible data from the last cluster is selected.

The above-described example of the method according to the first aspect may be summarized as follows:
Applying and combining the cluster rules determine a set of data regarded as associated with the same anatomic or physiological state of the patient. Each cluster of data for one anatomic or physiological state can be displayed as a cluster.
From all of the data of one cluster, initially only that data can be displayed that is most relevant for the user to use for further treatment planning or controlling a medical device by applying the set of filters provided.
A set of the displayed data can be selected into a temporary working set as proposal to the user to continue working with that set of data.

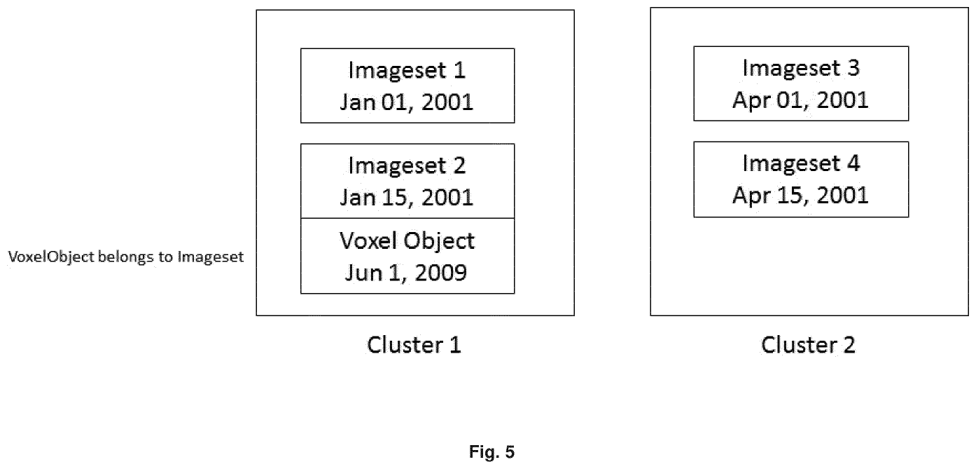

The patient data may comprise or consist of at least one of the following:
at least one of at least one imageset (e.g. CT, MR, Xray, PET), at least one screenshot or at least one videoclip;
treatment-related data associated with an imageset: e.g. at least one of at least one voxel object (which may have been added to an imageset as exemplified by FIG. 5), at least one trajectory, at least one points, at least one documents, or at least one fiber bundle;
at least one treatment plan which for example references a set of data—a user may have saved explicitly a treatment plan to store a set of references;
an auto-saved treatment plan, saved by e.g. another application working on the same set or subset of data to store the latest selection of a set of data that was used by a user without being actively created by the user (like a last working set).

Applications running on a system to provide all features may for example be the following:
A data source which provides patient data on request by e.g. an application such as a Brainlab application based on patient information.
A temporary working set provided and managed by the data source. Applications, e.g. Brainlab applications, can access the temporary working set to read and write references to patient data to communicate these references to other applications
A program that executes the clustering, filtering and selection rules on data provided by the data source.

Further features of the method according to the first aspect and the above example of that method may for example be:
Processed data may not available at once. Data is coming into the system from the data source providing the patient data one after the other and is processed immediately when it comes into the system. The order is arbitrary. Already available data is transferred from the data source, which takes some seconds to minutes. Data can come into the system any time later and is retrieved from the data source when available.
Imagesets (including screenshots) are ordered by creation date. External triggers may lead to a new cluster being created. Initially, there exists one cluster created with the first imageset that is processed.
A cluster has a time range defining the beginning and the end of the time range of the data that is inside the cluster. Cluster time ranges do not overlap.
An event describes a specific point in time. This can mean a new, additional cluster is created for all incoming data or an existing cluster is split into two, if the event has a time that is in between of the data that is already put into a cluster.
All clustering, filtering and selection rules are not a fixed set. New rules based on new information or knowledge that is available can be created.
Many of the rules can be created in a way that the behaviour regarding parameters that do not change the logic but the actual values that are used to take decisions (e.g. a time gap in days to create a new cluster) can be configured.
All parts of the method according to the first aspect (e.g. the clustering, filtering, and/or selection/pre-selection) can be created in a way that the parameters which describe the behaviour are determined automatically and change over time by analyzing the available set of data and previous usage behavior (machine learning). A machine learning algorithm can be applied to use all usage statistics and available information of the data to determine the quality of the result of the method. The quality of the result can be measured by e.g. time, the user needs to fulfil its task or the amount of specific user interaction that is required to fulfil the task. I.e. if a change in parameters gives a result that leads to shorter overall usage time and less clicks to fulfil the complete data selection task, the change can be considered as quality-increasing.
The method according to the first aspect aims to find data associated with the same anatomic or physiological state and filters and selects specific data within this set. The anatomic or physiological state is currently to use the best known criteria to decide that the set of data is relevant for the user. The overall goal is to select that set of data that is most relevant for the user.

In one example, the result of the mechanism to cluster, filter (and preselect) data is the input for a next workflow step which in examples may be described as follows:
A robotic application such as a robotic arm may use the selected data and determines if it contains a trajectory object. The arm can position itself according to the direction of the trajectory.
A positioning system for radiation treatment (e.g. ExacTrac) may position the patient for treatment using the imagesets of the result as reference images.
An image-guided navigation system for surgery uses the imagesets of the result data and displays these to the user.
Surgery Planning/Navigation
Deep Brain Stimulation
Transcranial Magnetic Stimulation
Storage and/or linking in external data management software

An aspect of the method according to the first aspect is to cluster (i.e. to group data in a way that it is visibly associated and separated from other clustered data) available patient data (imagesets and other treatment related data, e.g. planned objects, trajectories) in a way that each cluster of data describes one specific anatomic or physiological state of the patient.

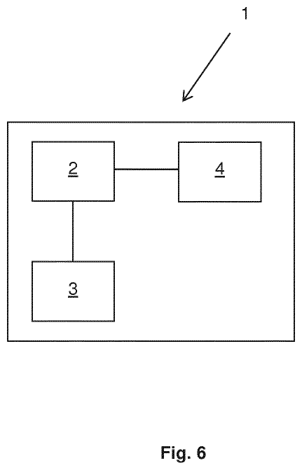

FIG. 6 is a schematic illustration of the medical system 1 according to the fifth aspect. The system is in its entirety identified by reference sign 1 and comprises a computer 2, an electronic data storage device (such as a hard disc) 3 for storing at least the patient data and a medical device 4 (such as a radiation treatment apparatus). The components of the medical system 1 have the functionalities and properties explained above with regard to the fifth aspect of this disclosure.

1.-20. (canceled)

21. A computer-implemented method, comprising:
acquiring patient data which describes medical information about a patient;
acquiring cluster rule data which describes at least one clustering rule for clustering the patient data;
determining cluster data based on the patient data and the cluster rule data, wherein the cluster data describes at least one cluster defining a clustering of the medical information for at least one anatomic or physiological state of the patient;
acquiring filter rule data which describes at least one filter rule for filtering at least one of the cluster data or the patient data;
determining filtered data based on the cluster data and the filter rule data, wherein the filtered data describes a result of applying a filter rule to the medical information for at least one cluster described by the cluster data, wherein the result describes at least one of the following:
an indication as to whether the filter rule can be applied to the medical information;
an indication as to whether the medical information shall be removed from the cluster;
an indication as to whether the medical information shall be used in a medical environment including at least one of: deep brain stimulation, or transcranial magnetic brain stimulation, or controlling a medical device on the basis of the result of applying the filter rule.

22. The method according to claim 21, wherein each cluster describes an association of at least one strict subset of the medical information with one anatomic or physiological state.

23. The method according to claim 21, wherein the at least one clustering rule includes at least one rule for clustering the medical information based on at least one of the following criteria:
a point in time at which the medical information was generated;
a time interval between points in time at which subsets of the medical information were generated;
manual input by a user for defining a cluster extent;
a type of the medical information;
an anatomic region to which the medical information relates;
the anatomic or physiological state.

24. The method according to claim 21, wherein the anatomic or physiological state encompasses a characterization of the patient's state at a specific point in time in consideration of a pathology of the patient.

25. The method according to claim 24, wherein the pathology encompasses a tumour disease and the anatomic or physiological state is defined by at least one of a size of a tumour or the degree of metastasis spread or a physiological indicator of the patient.

26. The method according to claim 21, wherein the at least one filter rule includes at least one rule for filtering the medical information based on at least one of the following criteria:
a point in time at which the medical information was generated;
a pathology of the patient;
a clinical workflow of which the patient shall be the subject;
an anatomic region to which the medical information relates;
a medical imaging modality to which the medical information relates or with which it was generated;
a size of the digital dataset comprising the medical information;
manual user input.

27. The method according to claim 21, wherein the medical information comprises at least one of the following:
a screenshot or a video clip;
medical image information;
medical non-image information, including a voxel object defining a subset of medical image information;
treatment plan information usable for use in at least radiotherapy or surgery;
trajectory information describing a trajectory for placing an instrument relative to a patient's body;
patient documentation describing a medical state of the patient;
a point, including at least one of annotation points or a landmark;
a fusion or a registration.

28. The method according to claim 21, wherein
selection data is determined based on the filtered data, wherein the selection data describes a selection of the medical information clustered in the at least one cluster described by the cluster data for use in the medical environment.

29. The method according to claim 28, wherein
selection rule data is acquired which describes at least one selection rule for selecting the selection of the medical information clustered in the at least one cluster described by the cluster data,
wherein the selection data is determined based on the selection rule data.

30. The method according to claim 29, wherein the selection rule includes at least one rule for selecting the medical information to which the filter rule has been applied,
wherein the selection rule is suitable for selecting the medical information based on at least one of the following criteria:
a type of the medical information;
the point in time with which a cluster is associated;
an envisaged medical procedure to be carried out on the patient.

31. The method according to claim 21, wherein controlling the medical device on the basis of the result of applying the filter rule includes at least one of:
a beam source or patient support unit of a radiation treatment apparatus;
an imaging unit of a radiation treatment apparatus;
a robot for conducting a medical procedure;
a display device of an image-guided navigation system to display the medical information;
a deep brain stimulation electrode or a transcranial magnetic stimulation device.

32. The method according to claim 21, comprising a step in which
atlas data is acquired which describes an image-based model of an anatomical body part of the patient,
wherein the cluster data is determined based on the patient data and the atlas data.

33. A computer program which, when running on at least one processor of at least one computer or when loaded into the memory of at least one computer, causes the at least one computer to perform the method according to claim 21.

34. A non-transitory computer-readable program storage medium storing a computer program which, when executed on at least one processor of at least one computer, causes the at least one computer to perform the steps comprising:
acquiring patient data which describes medical information about a patient;
acquiring cluster rule data which describes at least one clustering rule for clustering the patient data;
determining cluster data based on the patient data and the cluster rule data, wherein the cluster data describes at least one cluster defining a clustering of the medical information for at least one anatomic or physiological state of the patient;
acquiring filter rule data which describes at least one filter rule for filtering at least one of the cluster data or the patient data;
determining filtered data based on the cluster data and the filter rule data, wherein the filtered data describes a result of applying a filter rule to the medical information for at least one cluster described by the cluster data, wherein the result describes at least one of the following:
an indication as to whether the filter rule can be applied to the medical information;
an indication as to whether the medical information shall be removed from the cluster;
an indication as to whether the medical information shall be used in a medical environment including at least one of: deep brain stimulation or transcranial magnetic brain stimulation, or controlling a medical device on the basis of the result of applying the filter rule.

35. A system, comprising:
at least one computer having at least one processor and associated memory, the memory having instructions that when executed, perform the steps of:
acquiring patient data which describes medical information about a patient;
acquiring cluster rule data which describes at least one clustering rule for clustering the patient data;
determining cluster data based on the patient data and the cluster rule data, wherein the cluster data describes at least one cluster defining a clustering of the medical information for at least one anatomic or physiological state of the patient;
acquiring filter rule data which describes at least one filter rule for filtering at least one of the cluster data or the patient data;
determining filtered data based on the cluster data and the filter rule data, wherein the filtered data describes a result of applying a filter rule to the medical information for at least one cluster described by the cluster data, wherein the result describes at least one of the following:
an indication as to whether the filter rule can be applied to the medical information;
an indication as to whether the medical information shall be removed from the cluster;
an indication as to whether the medical information shall be used in a medical environment including at least one of: deep brain stimulation or transcranial magnetic brain stimulation, or controlling a medical device on the basis of the result of applying the filter rule;
at least one electronic data storage device storing at least the patient data; and
a medical device for carrying out a medical procedure on the patient,
wherein the at least one computer is operably coupled to the at least one electronic data storage device for acquiring from the at least one data storage device at least the patient data, and the medical device for issuing a control signal to the medical device for controlling the operation of the medical device on the basis of the result of applying the filter rule to the medical information.

36. The system according to claim 35, wherein the medical device comprises
a radiation treatment apparatus comprising a treatment beam source and a patient support unit,
wherein the at least one computer is operably coupled to the radiation treatment apparatus for issuing a control signal to the radiation treatment apparatus for controlling, at least one of:
the operation of the treatment beam source, or
the position of the patient support unit.

37. The system according to claim 35, wherein the medical device comprises
a robot for conducting a medical procedure,
wherein the at least one computer is operably coupled to the robot for issuing a control signal to the robot for controlling the operation of the robot.
